(19) United States
(12) Patent Application Publication
(10) Pub. No.: US 2005/0025724 A1
(43) Pub. Date: Feb. 3, 2005

(54) Composition and method for dry nail polish repair

(76) Inventor: Robert Laggan, North Haledon, NJ (US)

Correspondence Address: MUSERLIAN, LUCAS AND MERCANTI, LLP, 475 PARK AVENUE SOUTH, 15TH FLOOR, NEW YORK, NY, 10016, US

(21) Appl. No.: 10/864,196

(22) Filed: Jun. 9, 2004

Related U.S. Application Data
(60) Provisional application No. 60/490,668, filed on Jul. 28, 2003.

(51) Int. Cl. A61K 7/42; A61K 7/04

(52) U.S. Cl. 424/59; 424/61

(57) ABSTRACT

A nail polish repair composition and top coat is disclosed containing one or more resins, one or more plasticizers, one or more solvents, no pigment, and as an essential ingredient acetone in an amount of about 5 to about 20 percent by weight of said composition. The composition is applied to a dry nail polish so as to form a liquefied mixture of the composition and the dry nail polish. The liquefied mixture is manipulated to spread the original nail polish over the damaged area of the nail to repair the damaged area. The top coat is applied onto a dry nail polish to form a top coat.

### CROSS-REFERENCE TO RELATED APPLICATIONS

This Application claims the priority of U.S. Provisional Application Ser. No. 60/490,668, filed Jul. 28, 2003.

### BACKGROUND OF THE INVENTION

1. Field of the Invention

The present invention relates to a composition and method for repairing a damaged dry nail polish on a polished nail. The composition is applied to the dry nail polish so as to form a liquefied mixture of the composition and the dry nail polish. The liquefied mixture is manipulated to spread the original nail polish over the damaged area of the nail to repair the damaged area.

2. Art Related to the Invention

Dried nail polish inevitably becomes chipped, cracked or otherwise damaged after the nail polish has been applied to a nail. A damaged nail polish results in an unattractive appearance both to the individual and to others.

Efforts to quickly repair the damaged nail polish have proved to be unsuccessful. For example, the simple addition of more nail polish to the damaged area causes the newly added nail polish to dry with a raised surface compared to the surface of the original nail polish. This raised appearance is easily visible and thus is not considered satisfactory. Most likely, it is not even possible to attempt such a procedure, since an individual may not have immediate access to the original nail polish, as the nails may have been prepared at a salon or at home.

Alternatively, the entire original nail polish could be removed and reapplied to achieve the desired finish. However, this option is labor intensive and often costly, as it may require a trip to the salon or the purchase of additional nail polish. In addition, it is not sufficient to repair only the damaged nail polish on a single finger or toe, since the repaired nail polish will usually not match the original nail polish on the remaining fingers or toes.

There is a need for an acceptable technique that quickly and easily repairs a damaged dry nail polish without the need to replace the entire nail polish on all fingers or toes.

### SUMMARY OF THE INVENTION

The present invention is a composition and method for repairing a damaged, dry nail polish on a polished nail. The present invention eliminates the disadvantages of applying additional nail polish to the damaged area and eliminates the need to reapply the entire nail polish on all fingers or toes.

Broadly, the composition of the present invention comprises one or more resins, one or more plasticizers, one or more solvents, no pigment, and as an essential ingredient acetone in an amount of about 5 to about 20 percent by weight of said composition.

The composition further comprises one or more resins are present in a combined amount between about 5 to about 25 percent by weight of the composition, said one or more plasticizers are present in a combined amount between about 0.5 to about 6 percent by weight of the composition and said one or more solvents, other than acetone, are present in a combined amount between about 55 to about 80 percent by weight of the composition.

The composition further comprises a UV absorber and dimethicone.

Broadly, the method of the present invention comprises:
a) applying a composition comprising one or more resins, one or more plasticizers, one or more solvents, no pigment, and acetone in an amount between 5 and 20 percent by weight to a portion of a damaged, dried nail polish to form a liquefied mixture of said composition and said nail polish;
b) brushing said liquefied mixture to spread said liquefied mixture over a damaged area; and
c) drying said liquefied mixture to remove said acetone and said solvents thereby causing said mixture to solidify.

The composition of the present invention is applied onto the damaged nail polish on a previously polished nail. The composition is specifically designed to dissolve the original nail polish thereby forming a liquefied mixture of the composition and the original nail polish. The liquefied mixture is redistributed over a desired area of the nail using a conventional device in order to restore the damaged area of the nail with a portion of the original nail polish. The liquefied mixture is allowed to dry once it is determined that the redistributed mixture has a smooth, even appearance over the entire portion of the nail. A repaired nail polish is thereby achieved by relocating a portion of the original nail polish to the damaged area of the nail polish.

### DETAILED DESCRIPTION OF THE INVENTION

The nail polish repair composition of the present invention utilizes a portion of the dried, original nail polish to repair a nearby damaged area of the nail polish. A dried portion of the nail polish is liquefied upon introduction of the composition thereby allowing for a redistribution of the dried portion of the nail polish to the damaged area of the nail polish. The original nail polish is thus placed in a maneuverable, fluid-like state by combination with the composition.

When applying the composition, agitation or mixing of the composition with or into the dry nail polish can be necessary in order to form the mixture. Such agitation or mixing is done with a brush applicator used to apply the composition.

The nail polish can easily be manipulated and maneuvered over the entire surface of the nail once the nail polish has been liquefied or dissolved using the composition. A nail polish brush or other suitable device can be used to spread the liquefied nail polish over a damaged area of the nail polish. This spreading of the original nail polish covers or buries the damaged area of the nail polish with the original nail polish. Thus, the original nail polish is stretched or expanded to cover or fill not only the initial, undamaged portion of the nail polish, but also the damaged area of the nail polish. Preferably, the liquefied nail polish is brushed over the entire surface of the nail to form a uniform, smooth nail polish surface.

The dissolved nail polish is allowed to dry once the damaged area of the nail polish has been covered and repaired in a satisfactory manner. Drying of the dissolved nail polish allows for the evaporation of the acetone and any other solvents that are present in the composition. Upon evaporation, the liquefied nail polish solidifies, thus revealing a hardened nail polish. The hardened nail polish resembles the original nail polish without the damaged area. Drying can be conducted in any conventional manner, such as fan drying or air drying.

After the nail polish has dried, the composition can be applied as a top coat over the entire polished nail to provide gloss to the nail. The composition can be applied one or two times to act as a top coat.

The chemical makeup of the composition will now be described. The following discussion makes separate reference to acetone and solvents, even though acetone may be considered a solvent. Thus, the amount of acetone and the amount of solvents as described are to be individually determined. The amount of acetone is not included in the amount of solvents.

The nail polish repair composition of the present invention is comprised of acetone, one or more resins, one or more plasticizers, one or more solvents and no pigment. Additionally, the composition of the present invention contains no methyl ethyl ketone (MEK).

Acetone is present in an amount between about 5 to about 20 percent by weight of the composition. Preferably, acetone is present in an amount between about 10 to about 20 percent by weight. More preferably, acetone is present in an amount between about 11 to about 15 percent by weight. An acetone level above the broad range results in clouding of the nail polish after addition of the composition. An acetone level below the broad range is insufficient for liquefication of the nail polish.

Some dry nail polishes are harder to liquefy that others. It is believed that increasing the acetone amount in the composition will assist in liquefying such nail polish.

The acetone evaporates upon drying of the liquefied mixture of the nail polish and the composition thereby allowing the nail polish to harden.

One or more resins are present in a combined amount between about 5 to about 25 percent by weight of the composition. Preferably, the resins are present in a combined amount between about 10 to about 20 percent by weight of the composition. The resins impart adhesion and provide a protective coating to the dried nail polish after combination with the composition. The resins also serve as a viscosity adjuster for the liquefied mixture of the nail polish and the composition. Thus, it is desirable to select a resin or combination of resins in order to achieve a desired viscosity. If the viscosity is not controlled, it will be difficult to brush the liquefied mixture to achieve a smooth, acceptable surface. The viscosity of the composition and mixture can be adjusted by controlling the molecular weight of the resin.

Suitable resins are cellulose esters, such as cellulose acetate butyrate produced by Eastman Chemicals, nitrocellulose, cellulose acetate or cellulose acetate propionate. In addition, resins such as acrylates copolymer and polyester resin can be used. It is preferable that the resin has a Tukon Hardness of greater than 14 Knoops. A Tukon Hardness less than this value results in a dried nail polish that is soft and easily damageable. The acetylation level of the cellulose acetate butyrate resin should be preferably between about 2 to about 17.5 percent, and more preferably between about 13.5 to about 17.5 percent. The butyryl level should be preferably between about 32.5 to about 53 percent, and more preferably between about 32.5 to about 38 percent.

One or more plasticizers are present in a combined amount of about 0.5 to about 6 percent by weight of the composition. Preferably, the plasticizer is present in a combined amount between about 1 to about 5 percent by weight of the composition. The plasticizers are added to reduce shrinkage and make the film flexible.

Examples of the plasticizers are trimethyl pentanyl diisobutyrate, sucrose benzoate, triphenyl phosphate, dibutyl phthalate, acetyl dibutyl citrate and acetyl tributyl citrate.

One or more solvents, in addition to the acetone previously mentioned, is also contained in the composition. The solvents are present in a combined amount of between about 55 to about 80 percent by weight of the composition, preferably between about 60 to about 70 percent by weight of the composition. The amount of solvent is primarily selected to reflect the amount of acetone present in the composition. The higher the acetone content, the less solvent is necessary to achieve the effects of the invention. The viscosity of the composition and mixture can also be adjusted by controlling the amount of the solvent. The term solvent as used in the specification and claims excludes acetone.

Preferable solvents are ethyl acetate, SDA 40 anhydrous, butyl acetate, isopropanol, ethyl alcohol and butyl alcohol. Naturally, other solvents can be selected as well.

The viscosity of the composition is suitably adjusted by controlling the resin and the solvent as noted above. Preferably, the viscosity of the composition is between about 25 to about 300 cps (centipoises), more preferably about 100 cps.

The composition preferably contains one or more UV absorbers. UV absorbers tend to slow the yellowing of the composition caused by UV radiation. The UV absorbers protect the composition during storage, protect the liquefied mixture during application, and protect the hardened nail polish after repair. Preferably, the UV absorbers are present in a combined amount between about 0.2 to about 0.6 percent by weight of the composition, more preferably between about 0.3 to about 0.5 percent by weight of the composition. Suitable UV absorbers are etocrylene and benzophenone 1.

The composition also preferably contains dimethicone. Dimethicone is a nonvolatile silicone component that improves the flow characteristics of the liquefied mixture during spreading of the liquefied mixture. Dimethicone if present in an amount between about 0.05 to about 0.15 percent by weight of the composition, preferably between about 0.08 to about 0.10 percent by weight of the composition. Dimethicone is added to provide good top coat characteristics and can be absent from the composition if another composition is used as a top coat.

The formation of the composition is not particularly limited. However, it has been found that the viscosity of the composition can be controlled by slowly adding resins to a combined mixture of the solvents.

The composition is preferably a transparent liquid, containing no pigments or other colors typically found in nail polish. The essential purpose of the composition is to dissolve the nail polish in order to allow the nail polish to be painted in a conventional manner throughout the surface of the nail. The transparent nature of the composition allows for combination with all commercial nail polishes without interference with the original color of the nail polish.

The composition of the present invention can also be used as a top coat for a polished nail. The composition is used as a top coat by applying the composition to the dried nail polish as the last coat in a conventional manner. It can be suitably applied multiple times to provide multiple top coats. The composition has dimethicone when used as a top coat.

### EXAMPLES

The following Examples are preferred embodiments of the present invention.

### Example 1

A nail polish repair composition was prepared by mixing acetone, a plurality of resins, a plurality of solvents, a plurality of plasticizers, a plurality of UV absorbers and dimethicone. The weight percentages of each of these compounds is illustrated in Table 1.

### Example 2

A nail polish repair composition was prepared in the same manner as Example 1, except that the amount of acetone was varied. The weight percentage of each of the compounds of Example 2 is illustrated in Table 1.

TABLE 1

| COMPONENT | EXAMPLE 1 (% w/w) | EXAMPLE 2 (% w/w) |
|---|---|---|
| Ethyl Acetate | 45.27 | 34.24 |
| Acetone | 11.42 | 14.29 |
| SDA 40 Anhydrous | 10.28 | 12.86 |
| Butyl Acetate | 10.28 | 12.86 |
| Cellulose Acetate | 7.99 | 9.44 |
| Butyrate | | |
| Acrylate Copolymer | 7.54 | 9.43 |
| Trimethyl Pentanyl | 0.00 | 2.46 |
| Diisobutyrate | | |
| Sucrose benzoate | 0.00 | 1.70 |
| Triphenyl Phosphate | 0.00 | 0.86 |
| Polyester Resin | 3.84 | 0.00 |
| Nitrocellulose | 1.33 | 0.86 |
| Acetyl Tributyl Citrate | 1.07 | 0.00 |
| Isopropanol | 0.56 | 0.37 |
| Etocrylene | 0.34 | 0.43 |
| Benzophonone 1 | 0.00 | 0.10 |
| Dimethicone | 0.08 | 0.10 |
| TOTAL | 100.00 | 100.00 |

1. A nail polish repair composition comprising one or more resins, one or more plasticizers, one or more solvents, no pigment, and as an essential ingredient acetone in an amount of about 5 to about 20 percent by weight of said composition.

2. The composition of claim 1, wherein said one or more resins are present in a combined amount between about 5 to about 20 percent by weight of the composition, said one or more plasticizers are present in a combined amount between about 0.5 to about 6 percent by weight of the composition and said one or more solvents are present in a combined amount between about 55 to about 80 percent by weight of the composition.

3. The composition of claim 1 further comprising a UV absorber.

4. The composition of claim 1 further comprising dimethicone.

5. A method for repairing a damaged nail polish on a nail comprising:
a) applying a composition comprising one or more resins, one or more plasticizers, one or more solvents, no pigment, and acetone in an amount between 5 and 20 percent by weight to a portion of said nail polish to form a liquefied mixture of said composition and said nail polish;
b) brushing said liquefied mixture to spread said liquefied mixture over a damaged area; and
c) drying said liquefied mixture to remove said acetone and said solvents thereby causing said mixture to solidify.

6. The method of claim 5, wherein said one or more resins are present in a combined amount between about 5 to about 20 percent by weight of the composition, said one or more plasticizers are present in a combined amount between about 0.5 to about 6 percent by weight of the composition and said one or more solvents are present in a combined amount between about 55 to about 80 percent by weight of the composition.

7. The method of claim 5, wherein said composition further comprises a UV absorber.

8. The method of claim 5, wherein said composition further comprises dimethicone.

9. A nail polish top coat comprising:
one or more resins, one or more plasticizers, one or more solvents, no pigment, and as an essential ingredient acetone in an amount of about 5 to about 20 percent by weight of said composition; and dimethicone.

10. A method for adding a top coat to a polished nail comprising:
a) applying a composition comprising one or more resins, one or more plasticizers, one or more solvents, no pigment, and as an essential ingredient acetone in an amount of about 5 to about 20 percent by weight of said composition to a dry nail polish; and
b) drying said composition to provide a top coat to said dry nail polish.
